(19) United States
(12) Patent Application Publication
(10) Pub. No.: US 2007/0275920 A1
(43) Pub. Date: Nov. 29, 2007

(54) Method for Chromatographic Separation of a Nucleic Acid Mixture

(76) Inventors: Markus Muller, Dormagen (DE); Jorg Hucklenbroich, Remscheid (DE); Lothar Breitkopf, Bonn (DE)

Correspondence Address: MCDERMOTT WILL & EMERY LLP, 600 13TH STREET, N.W., WASHINGTON, DC, 20005-3096, US

(21) Appl. No.: 10/587,892

(22) PCT Filed: Jan. 25, 2005
(86) PCT No.: PCT/EP05/00693

§ 371 Date: Jun. 18, 2007

(30) Foreign Application Priority Data
Jan. 29, 2004 (EP) 04001864.0

(51) Int. Cl. A61K 48/00; A61P 43/00; C07H 21/04

(52) U.S. Cl. 514/44; 536/25.4

(57) ABSTRACT

The invention relates to a method for chromatographic separation of a nucleic acid mixture, particularly to the separation and purification of plasmid DNA from other components of the nucleic acid mixture, particularly other nucleic acids. The inventive method is particularly characterized in that plasmid DNA can be separated without adding ribonucleases of contaminating RNA and is also characterized by the use of low-cost environmentally-friendly components. Said parameters enable the inventive method to also be used for the large-scale production of plasmid DNA. The invention also relates to the use of plasmid DNA obtained by means of the inventive method in the production of an agent containing plasmid DNA and which can be used in gene therapy and genetic vaccination.

The subject matter of the present invention is a method for the chromatographic separation of a nucleic acid mixture, in particular for the separation and purification of plasmid DNA from other components of the nucleic acid mixture, especially other nucleic acids. The method of the invention is characterised in particular in that plasmid DNA can be separated from contaminating RNA without the addition of ribonucleases as well as by the use of cost-effective and environmentally compatible components. These parameters also allow the use of this method for the production of plasma DNA on a large scale. Moreover the present invention comprises the use of the plasmid DNA isolated by this method for the preparation of an agent containing plasmid DNA for use in gene therapy and genetic vaccination.

A fundamental problem in the purification of plasmids is the removal of other nucleic acid species from the product. This problem arises mainly in areas in which particularly pure plasmid DNA preparation is required, for example in the use of plasmid DNA in gene therapy. The other nucleic acid species mentioned are mainly the different RNAs, but also include genomic DNA and ssDNA (single stranded), etc. A particular difficulty is the removal of RNA. A technique known from the state of art is the removal of RNA with the aid of ribonucleases. The RNA is degraded to ribonucleotides by means of the ribonucleases and can be far more readily removed from the plasmid DNA in a subsequent chromatographic separation procedure. The considerable disadvantage of this method is the use of an RNase, which usually represents a foreign protein. The RNase is isolated from animal material, usually from cattle. In the preparation of parenteral therapeutic agents for administration to humans in particular the addition of animal proteins in production processes is to be excluded owing to a possible contamination of the product with bacterial, viral or proteinogenic pathogens. This applies especially to bovine proteins owing to the problem of BSE.

Moreover, the use of RNase and alcohol-containing buffers represent a large cost factor. Particularly in the production of plasmid DNA on a large scale, that is in the ranges of about >2 g and above, this is a cost factor that is not to be underestimated. The use of alcohols also adds a significant burden for the employees involved and for the environment.

A general problem in the purification of nucleic acids from prokaryontic, and also from eukaryontic cells, is the lysis of cells that has to be carried out initially in order to bring about the release of the nucleic acids. In the method of the invention the alkaline lysis described in principle by Birnborn and Dohly (Nucl. Acids Res. 7, pp. 1513 - 1522; 1979) is preferred, but not restricted to it. Further possibilities are the lysis by heat or lysis in the presence of detergents. Lysis by high pressure (French Press) has proved to be unsuitable as very small fragments of genomic DNA arise from the high shear forces that result and are essentially no longer separable from the plasmid DNA.

Chromatographic procedures for the purification of nucleic acids from such lysates are known from the state of art. Here two distinct procedures are to be differentiated in general. One known from the state of art is the method of Gillespie and Vogelstein (Proc. Natl. Acad. Sci., USA, 76 pp. 615-619; 1979). In this method purification of the nucleic acids takes place by binding to silica gel or diatomaceous earth in the presence of chaotropic salts such as, for example, GuHCl, NaI, etc. Unlike anion exchangers, the binding of DNA occurs in the presence of high salt concentrations, whereas elution is carried out in the presence of low salt concentration. Since in this method the binding of nucleic acids follows the “all or nothing” principle, a quantitative separation of RNA, ssDNA and proteins is not possible. Therefore DNA samples obtained by means of this method are unsuitable for use in gene therapy owing to their contamination with RNA and proteins.

The second method to be cited is purification by ion exchangers as described in EP 0268 946. In this case cells, for example bacteria, are disintegrated preferably by alkaline lysis. The cellular proteins and genomic DNA are separated by detergents and subsequent centrifugation. The supernatants comprising plasmid DNA thus obtained are called cleared lysates. The cleared lysate is purified on an anion exchange column (e.g. QIAGEN®, QIAGEN GmbH, Hilden, Germany) when quantitative separation of RNA and ssDNA takes place.

The technical problem forming the basis of the method of the invention is the purification of plasmid DNA from a mixture of nucleic acids and the improvement of the separation of contaminants such as RNA, ssDNA and genomic DNA without the use of an RNase. A further task forming the basis of the invention is also to provide a method that allows purification of plasmids that is cost-effective and environmentally friendly, even on a large scale.

Surprisingly the technical problem forming the basis of the invention was solved by a method as described in the claims. In the method of the invention the separation of plasmid DNA from the other components of the nucleic acid mixture, in particular other nucleic acid species,
a) as appropriate the nucleic acid mixture is adjusted with one or more alkali salts and/or alkaline earth salts in aqueous solution to a conductance that is equivalent to a conductance of 70 mS to 95 mS at a pH of 4.8 to 5.4 at a temperature of 20° C., and
b) the nucleic acid mixture is brought into contact with a chromatographic stationary phase,
a) the stationary phase is then washed at least once with a solution comprising an alkali salt in a concentration range of 900 mM to 1800 mM based on a pH of 7 to 7.4 and/or an alkaline earth salt in a concentration range of 100 mM to 240 mM based on a pH of 7 to 7.4 and
c) the plasmid DNA bound to the chromatographic stationary phase is subsequently eluted with a solution comprising an alkali salt in a concentration of 1300 mM or higher based on a pH of 7 to 7.4 and/or an alkaline earth salt in a concentration of 270 mM or higher based on a pH of 7 to 7.4.

To isolate the nucleic acid mixture the cells, which may be prokaryontic or eukaryontic, are first lysed. This can be carried out in the manner described above. In the method of the invention the alkaline lysis described in principle by Birnborn and Dohly (Nucl. Acids Res. 7, pp. 1513-1522; 1979) is preferred, but not limited to it. Further possibilities are the lysis by heat or lysis in the presence of detergents. Lysis by high pressure (French Press) has proved to be unsuitable as very small fragments of genomic DNA arise from the high shear forces that result and which are essentially no longer separable from the plasmid DNA.

A nucleic acid mixture within the meaning of the invention can be thereby a cell lysate or equally a pre-purified or cleared lysate, but can also be an artificial mixture in which plasmid DNA is contaminated with at least one further nucleic acid species and if appropriate other contaminants. In a preferred embodiment of the method of the invention the nucleic acid mixture concerned is a prokaryontic cleared lysate.

The method of the invention ensures the chromatographic separation of the described contaminants and provides a plasmid DNA that fulfils the demands for purity for use in gene therapy or genetic vaccination. The person skilled in the art understands chromatography as a generic term for the physical-chemical separation of substance mixtures on the basis of their different distribution between a stationary phase and a mobile phase. In the representative method here an ion exchange material is used for the separation of the plasmid DNA from the contaminants. Surprisingly the commercially available material QIAGEN® (QIAGEN GmbH, Hilden, Germany) in particular has proved suitable for use in the method of the invention. This material allows a very efficient separation of RNA as well as, for example, ssDNA, from plasmid DNA by means of the method of the invention. Under the conditions described here in more detail RNA and ssDNA elute in a distinct peak that lies very far from the equally distinct peak of plasmid DNA in the method of the invention. The danger of co-elution of plasmid DNA and RNA and/or ssDNA is thus considerably reduced in comparison to methods known from the state of art.

The chromatographic stationary phase known under the name QIAGEN® (QIAGEN GmbH, Hilden, Germany) is a modified porous inorganic material. Suitable as inorganic supports for a chromatographic stationary phase in the method of the invention are silica gel, diatomaceous earth, glass, aluminium oxide, titanium oxide, zirconium oxide, hydroxyapatite, and as organic stationary phase those such as dextran, agarose, acrylamide, polystyrene resin or copolymers from the monomeric components of the named materials.

The anion exchanger that is preferably used in the method of the invention is obtained, for example, by the reaction in a first step of one of the aforementioned stationary phase materials with a silanisation reagent of the general structure I
R¹R²R³SiR⁴ (1)

wherein
R¹ is an alkoxy residue with 1 to 10 C atoms, especially —OCH₃, —OC₂H₅ or —OC₃H₇, or a halogen atom, especially —Cl, or a dialkylamino group with identical or different alkyl residues with 1 to 6 C atoms;
R² and R³ independently of one another are hydrocarbon residues with 1 to 10 C atoms, especially —CH₃, —C₂H₅ or —C₃H₇, or an alkoxy residue with 1 to 10 C atoms, especially —OCH₃, —OC₂H₅ or —OC₃H₇, or a halogen atom or an alkyl residue with 4 to 20 carbon atoms interrupted by at least one oxygen atom or amino group, wherein this residue can also be substituted once or several times by halogen, cyano, nitro, amino, monoalkylamino, dialkylamino, hydroxy or aryl;
R⁴ is a hydrocarbon chain with 1 to 20 C atoms or an alkyl residue interrupted by at least one oxygen atom or amino groups, wherein this residue can also be substituted once or several times by halogen, cyano, nitro, amino, monoalkylamino, dialkylamino, alkoxy, hydroxy, aryl and/or epoxy, especially

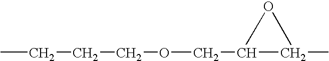

followed by a second step wherein the stationary phase modified in the first step is reacted with a reagent of the general structure II
X—R—Y (II)

wherein
X is an amino-, hydroxy-, epoxy group or a halogen atom,
R is a hydrocarbon chain with 2 to 20 C atoms or an alkyl residue interrupted by at least one oxygen atom or amino group, where in this residue can also be substituted once or several times by halogen, cyano, nitro, amino, monoalkylamino, dialkylamino, alkoxy, hydroxy, aryl and/or epoxy,
Y is a hydrocarbon residue with anion exchange forming functional groups with 1 to 10 C atoms that can be substituted once or several times by amino-, monoalkylamino-, dialkylamino-, trialkylammonium, such as is also described in EP 0 743 949, page 4 to 5, to which content-wise reference is also made here.

In a preferred embodiment the nucleic acid mixture with adjusted one or more alkali salts and or alkaline earth salts in aqueous solution is to a conductance that is equivalent to a conductance of 70 mS to 95 mS at a pH of 4.8 to 5.4 at a temperature of 20° C. in accordance with the aforementioned facultative step a) of the above-described method of the invention. The person skilled in the art is aware that the conductance of a salt-containing solution can vary notably depending upon the respective temperature and pH and on the basis of the laws known to him can undertake a corresponding adjustment of the conductance by variation in the temperature and/or pH so that an implementation of the method of the invention is possible without any problem.

The salts preferably used in the method of the invention are alkali salts, that is salts with which the cationic components or part of the cationic components come from an element of the first main group of the periodic system of elements, and/or alkaline earth salts, that is salts with which the cationic components or part of the cationic components come from an element of the second main group of the periodic system of elements. Particularly preferred in the case of the alkali salts are alkali halides, and in the case of the alkaline earth salts alkaline earth halides. Particularly preferred is the use of the alkali halides KCl, NaCl, CsCl and/or LiCl as well as the alkaline earth halide CaCl₂. As alternative to the alkali or alkaline earth salts an ammonium salt (pseudoalkali salt) can be used in the method of the invention, preferably the ammonium salt of a carboxylic acid, more preferably ammonium acetate. Most preferably the salts used are KCl and/or NaCl. In addition to the individual salts mixtures of different alkali salts and/or alkaline earth salts can also be used in the method of the invention.

In the aforementioned washing step c) for elution of the contaminants alkali salts are used in a concentration range of 900 mM to 1800 mM based on a pH of 7 to 7.4 and/or alkaline earth salts in a concentration range of 100 mM to 240 mM based on a pH of 7 to 7.4. Fundamentally all aqueous solutions that appear sensible to a person skilled in the art can be used for the washing stage, for example buffered solutions such as, for example but not restricted to, Tris, potassium acetate, borate or MOPS buffered systems, or alternatively unbuffered systems, that is the salts are merely dissolved in water. Different pH values can potentially also result from different buffer systems, or they can be adjusted. The concentration ranges selected here relate here to a pH value of 7 to 7.4, generally the pH value of the washing solution can be varied within the aforementioned pH range. It is known to the person skilled in the art that during a change in the pH value of such a washing solution the concentration of the salts contained therein must also be changed to achieve the same effect, in this case the elution of the contaminants, that means, during implementation of the method there is a shift in the elution points of the contaminants (e.g. RNA) and plasmid DNA at the same pH value of the washing and elution solution, but not to a shift of the relationship of the elution points, which means that the separation in elution points of the different nucleic acid species remains advantageously the same. The parameters necessary for this can be undertaken by the person skilled in the art on the basis of his technical know-how without inventive input. The method of the invention comprises at least one washing step, but several washing steps, in a number that appears meaningful to a person skilled in the art, can also be carried out, also with washing buffers of the invention that differ among one another.

In a preferred embodiment of the invention at least one washing step is carried out with a solution comprising KCl in a concentration range of 1100 mM to 1800 mM based on a pH of 7 to 7.4, especially preferred at least one washing step is carried out with a solution comprising KCl in a concentration range of 1300 mM to 1700 mM based on a pH of 7 to 7.4.

In a further preferred embodiment of the invention at least one washing step is carried out with a solution comprising NaCl in a concentration range of 950 mM to 1200 mM based on a pH of 7 to 7.4, especially preferred at least one washing step is carried out with a solution comprising NaCl in a concentration range 1100 mM to 1150 mM based on pH of 7 to 7.4.

In the aforementioned elution step d) for elution of the plasmid DNA alkali salts are used in a concentration range of 1300 mM or higher based on a pH of 7 to 7.4 and/or alkaline earth salts in a concentration of 270 mM or higher based on a pH of 7. to 7.4. In analogy to the washing step, all aqueous solutions that appear sensible to a person skilled in the art can be used in principle for the washing stage, for example buffered solutions such as, for example but not restricted to, Tris, potassium acetate, borate or MOPS buffered systems, or alternatively unbuffered systems, that is the salts are merely dissolved in water. Different pH values can potentially also result from different buffer systems, or they can be adjusted. The concentration ranges selected here relate here to a pH value of 7 to 7.4, generally the pH value of the elution solution can be varied within the aforementioned pH range. It is known to the person skilled in the art that for a change in the pH value of such a elution solution the concentration of the salts contained therein must also be changed to achieve the same effect, in this case the elution of the contaminants, that means, during implementation of the method there is a shift in the elution points of the contaminants (e.g. RNA) and plasmid DNA at the same pH value of the washing and elution solution, but not to a shift of the relationship of the elution points, which means that the separation in elution points of the different nucleic acid species remains advantageously the same. The parameters necessary for this can be undertaken by the person skilled in the art on the basis of his technical know-how without inventive input.

In a preferred embodiment the elution step is carried out with a solution comprising KCl in a concentration of 1900 mM or higher based on a pH of 7 to 7.4. The upward concentration of KCl is only limited by its solubility in the solvent used.

In a further preferred embodiment the elution step is carried out with a solution comprising NaCl in a concentration above 1300 mM or higher based on a pH of 7 to 7.4. The upward concentration of NaCl is only limited by its solubility in the solvent used.

The adjustment of the conductance of the nucleic acid mixture before bringing the nucleic acid mixture into contact with the chromatographic stationary phase is carried out as already mentioned above also with alkali salts and/or alkaline earth salts. In a particularly preferred embodiment the nucleic acid mixture is adjusted with KCl to a conductance which is equivalent to a conductance of 70 mS to 80 mS at a pH of 4.8 to 5.4 and a temperature of 20° C., most particularly preferred to a conductance which is equivalent to a conductance of 70 mS to 80 mS at a pH of 4.8 to 5.4 and at a temperature of 20° C. In further particularly preferred embodiment the nucleic acid mixture is adjusted with NaCl to a conductance which is equivalent to a conductance of 70 mS to 95 mS at a pH of 4.8 to 5.4 at a temperature of 20° C., most particularly preferred to a conductance which is equivalent to a conductance 85 mS to 95 mS at a pH of 4.8 to 5.4 and at a temperature of 20° C.

In one preferred embodiment in the method of the invention in at least one of the washing steps of the chromatographic stationary phase identified by c) the alkali halide KCl is used.

The method of the invention is preferably carried out at room temperature. In the present case room temperature means that the method is carried out under normal process conditions corresponding approximately to a range of 18° C. to 25° C. Basically the method can be carried out at all temperatures that appear useful to the person skilled in the art.

Preferably the method of the invention is suitable for the purification of plasmid DNA. Surprisingly and advantageously it has emerged that plasmids of the most different size show no significant difference in the elution point, that is, in the salt concentrations at which an elution of the plasma DNA from the chromatographic stationary phase takes place. An adaptation of the parameters of the method, for example salt concentrations or pH value, to different plasmid sizes is thus not necessary.

Since with the subject matter of the invention a method is provided in which plasmid can be obtained on a large scale for the production of a plasma DNA-containing agent for use in gene therapy or genetic vaccination, endotoxin removal can be incorporated advantageously into the method totally without problems. Almost all of the methods known from the state of art can be used for this. For example the cleared lysate can be treated with an endotoxin removal buffer known from the state of art (e.g. comprising Triton X 100, Triton X 114, Polymyxin, etc.) and further used without change in the present method of the invention.

### FIGURES

FIG. 1:

The extinction at 254 nm of the throughput of an HPLC column packed with QIAGEN® chromatographic stationary phase is plotted against the KCl concentration. The elution of different nucleic acid species with increasing KCl concentration is illustrated. The experimental conditions are described in more detail in Example 2.

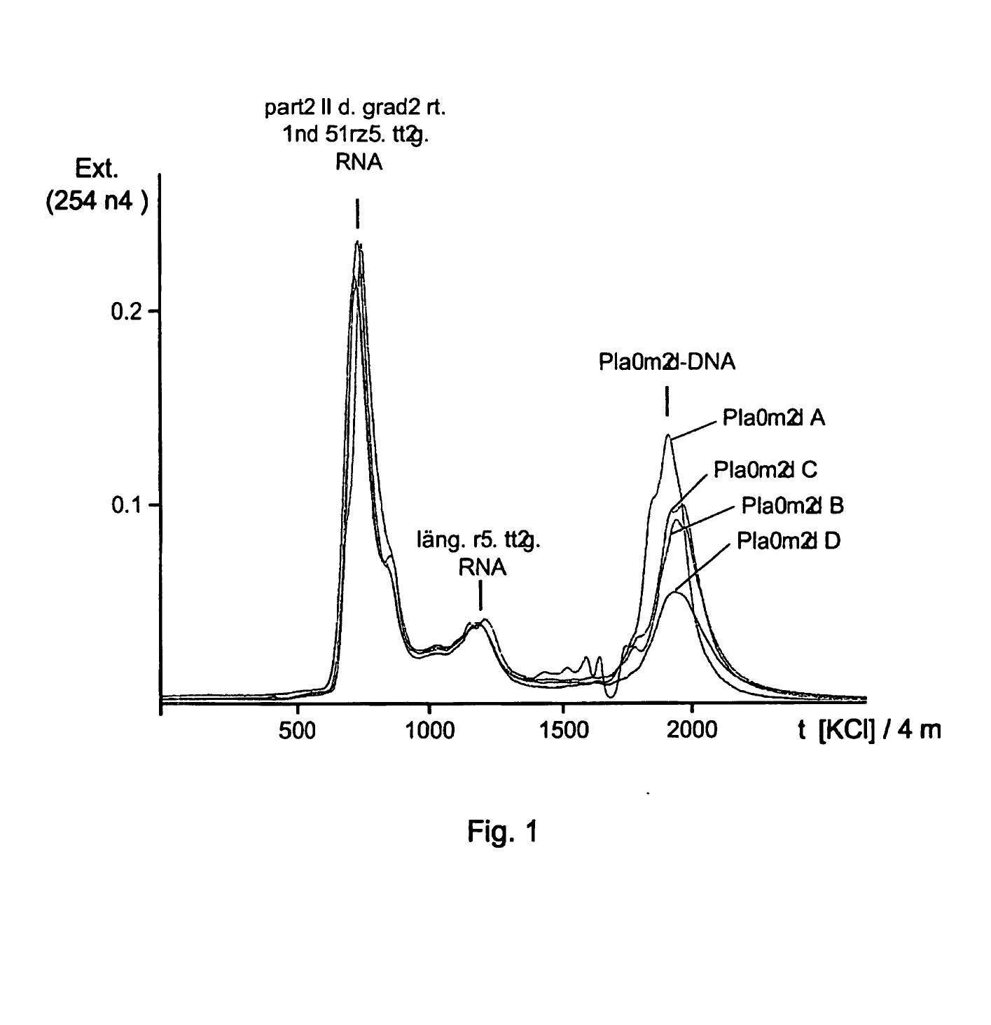

### LEGENDS TO FIG. 1

Peak 1—Partially degraded and short-chain RNA

Peak 2—long-chain RNA

Peak 3—Plasmid DNA

### EXAMPLES

### Example 1

### Purification of PCMVβ from E.coli DH5α

1 Kg biomass comprising pCMVP plasmid was obtained by centrifugation from a 30 L overnight fermentation culture of E.coli DH5α. The biomass was re-suspended in 15 L of a resuspension buffer (10 mM EDTA; 50 mM Tris/HCl pH 8) and then incubated with 15 L lysis buffer (200 mM NaOH; 1% (w/v) SDS) for 10 minutes at room temperature. Subsequently 15 L of a neutralisation buffer (3 M potassium acetate, pH 5.5) were added. The precipitate formed in this step (proteins, membrane components, genomic DNA, etc.) was then rejected. The thus pre-cleared lysate was then filtered, when a cleared lysate was produced. The cleared lysate subsequently had a pH of 5.2 and was adjusted with 3 M KCl to a conductance of 80 mS at a temperature of 20° C.

A chromatography column was packed with QIAGEN® chromatography stationary phase (column volume ca 7 L) and equilibrated with 10 column volumes of an equilibration buffer (20 mM potassium acetate) at a flow rate of 3.3 cm/min. The cleared lysate was loaded onto the column after equilibration of the chromatographic material, the run was carried out with a flow rate of 1.1 cm/min. Subsequently 5 column volumes of equilibrium buffer (20 mM potassium acetate) was passed through the column at a flow rate of 3.3 cm/min.

Immediately afterwards the column was washed with 10 column volumes of a KCl solution (1350 mM KCl; 50 mM Tris/HCl, pH 7.2) at a flow rate 3.3 cm/min. Following this the plasmids were eluted with a column volume of an elution buffer (1600 mM NaCl; 50 mM Tris/HCl, pH 7.2). After subsequent ultra-/diafiltration and final sterile filtration ca. 400 mg pCMVP was obtained.

### Example 2

In 4 different batches each of 600 μg of a purified plasmid (1: plasmid A [3266 bp]; 2: plasmid B [7200 bp];3: plasmid C [7687 bp]; 4: plasmid D [19535 bp]) together with in each case 600 μg purified RNA (E.coli HB101) were dissolved in 5 ml 60 mM potassium acetate.

An HPLC column (column volume 4.4 ml) was packed with QIAGEN® chromatographic stationary and equilibrated with 2 column volumes (flow rate 1 ml/min) equilibration buffer (60 mM potassium acetate). Next with 4 individual HPLC columns, freshly packed each time, the 5 ml of the plasmid-RNA mixtures were loaded onto the column at 1 ml/min. The columns were then rinsed with 3 column volumes of 60 mM potassium acetate.

A Tris buffer was passed through the column in a continuous gradient (50 mM Tris/HCl; pH 7.2; gradient 0 to 3 M KCl) and the elution of DNA and RNA measured with a photometer (extinction measurement at 254 nm). In this way it was possible to demonstrate that partially degraded and short chain RNA is eluted in a distinct peak (maximum at 780 mM KCl), followed by a somewhat diffuse peak of longer chain RNA (maximum at 1120 mM KCl, end of elution at 1310 mM KCl). The elution of the plasmid DNA reached a maximum at 1900 mM KCl and ended at 2150 mM KCl. The results are illustrated in superimposed traces in FIG. 1. It is clear that the plasmids are advantageously eluted independently of their size.

1. Method for the chromatographic separation of a nucleic acid mixture wherein plasmid DNA is separated from other components of the mixture, especially other nucleic acids, characterised in that
b) as appropriate the nucleic acid mixture is adjusted with one or more alkali salts and/or alkaline earth salts in aqueous solution to a conductance that is equivalent to a conductance of 70 mS to 95 mS at a pH of 4.8 to 5.4 at a temperature of 20° C., and
b) the nucleic acid mixture is brought into contact with a chromatographic stationary phase,
c) the stationary phase is then washed at least once with a solution comprising an alkali salt in a concentration range of 900 mM to 1800 mM based on a pH of 7 to 7.4 and/or an alkaline earth salt in a concentration range of 100 mM to 240 mM based on a pH of 7 to 7.4, and
d) the plasmid DNA bound to the chromatographic stationary phase is subsequently eluted with a solution comprising an alkali salt in a concentration of 1300 mM or higher based on a pH of 7 to 7.4 and/or an alkaline earth salt in a concentration of 270 mM or higher based on a pH of 7 to 7.4.

2. Method as described in claim 1, characterised in that the alkali salt is an alkali halide and the alkaline earth salt is an alkaline earth halide.

3. Method as described in claim 2, characterised in that the alkali halide is NaCl, KCl, CsCl and/or LiCl and the alkaline earth halide is CaCl₂.

4. Method as described in claim 1, characterised in that the nucleic acid mixture is adjusted with KCl to a conductance that is equivalent to a conductance of 70 mS to 85 mS at a pH of 4.8 to 5.4 and a temperature of 20° C.

5. Method as described in claim 4, characterised in that the nucleic acid mixture is adjusted with KCl to a conductance that corresponds to a conductance of 70 mS to 80 mS at a pH of 4.8 to 5.4 and a temperature of 20° C.

6. Method as described in claim 1, characterised in that the nucleic acid mixture is adjusted with NaCl to a conductance that corresponds to a conductance of 70 mS to 95 mS at a pH of 4.8 to 5.4 and a temperature of 20° C.

7. Method as described in claim 6, characterised in that the nucleic acid mixture is adjusted with NaCl to a conductance that corresponds to a conductance of 85 mS to 95 mS at a pH of 4.8 to 5.4 and a temperature of 20° C.

8. Method according to claim 1, characterised in that the washing step/s from step b) of claim 1 is/are carried out with a solution comprising KCl in a concentration range of 1100 mM to 1800 mM based on a pH of 7 to 7.4.

9. Method according to claim 8, characterised in that the washing step/s from step b) of claim 1 is/are carried out with a solution comprising KCl in a concentration range of 1300 mM to 1700 mM relating to a pH of 7 to 7.4.

10. Method according to claim 1, characterised in that the washing step/s from step b) of claim 1 is/are carried out with a solution comprising KCl in a concentration range of 950 mM to 1200 mM based on a pH of 7 to 7.4.

11. Method according to claim 10, characterised in that the washing step/s from step b) of claim 1 is/are carried out with a solution comprising NaCl in a concentration range of 1100 mM to 1150 mM based on a pH of 7 to 7.4.

12. Method as described in claim 1, characterised in that the elution step from step c) from claim 1 is carried out with a solution comprising KCl in a concentration of 1900 mM or higher based on a pH of 7 to 7.4.

13. Method as described in claim 1, characterised in that the elution step from step c) from claim 1 is carried out with a solution comprising NaCl in a concentration of 1300 mM or higher based on a pH of 7 to 7.4.

14. Method as described in claim 1, characterised in that the chromatographic stationary phase is an anion exchanger.

15. Method as described in claim 1, characterised in that silica gel, diatomataceous earth, glass, aluminium oxide, titanium oxide, zirconium oxide, hydroxy apatite, dextran, agarose, acrylamide, polystyrene resin or copolymers of the named materials are used as chromatographic stationary phase.

16. Method as described in claim 15, characterised in that the chromatographic stationary phase is obtainable by reaction of one of the stationary phase named in claim 15 in a first step with a silanisation reagent of the general structure I
R¹R²R³SiR⁴ (4)
wherein
R¹ is an alkoxy residue with 1 to 10 C atoms, especially —OCH₃, —OC₂H₅ or —OC₃H₇, or a halogen atom, especially —Cl, or a dialkylamino group with identical or different alkyl residues with 1 to 6 C atoms;
R² and R³ independently of one another are hydrocarbon residues with 1 to 10 C atoms, especially —CH₃, —C₂H₅ or —C₃H₇, or an alkoxy residue with 1 to 10 C atoms, especially —OCH₃, —OC₂H₅ or —OC₃H₇, or a halogen atom or an alkyl residue with 4 to 20 carbon atoms interrupted by at least one oxygen atom or amino groups, wherein this residue can also be substituted once or several times by halogen, cyano, nitro, amino, monoalkylamino, dialkylamino, hydroxy or aryl;
R⁴ is a hydrocarbon chain with 1 to 20 C atoms or an alkyl residue interrupted by at least one oxygen atom or amino group, whereby this residue can also be substituted one ore several times with halogen, cyano, nitro, amino, monoalkylamino, dialkylamino, alkoxy, hydroxy, aryl and/or epoxy, especially 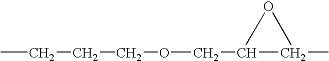
followed by a second step wherein the stationary phase modified in the first step is reacted with a reagent of the general structure II
X—R—Y (II)
wherein
X is an amino-, hydroxy-, epoxy group or a halogen atom,
R is a hydrocarbon chain with 2 to 20 C atoms or an alkyl residue interrupted by at least one oxygen atom or amino group, where in this residue can also be substituted once or several times by halogen, cyano, nitro, amino, monoalkylamino, dialkylamino, alkoxy, hydroxy, aryl and/or epoxy,
Y is a hydrocarbon residue with anion exchange forming functional groups with 1 to 10 C atoms that can be substituted once or several times by amino-, monoalkylamino-, dialkylamino-, trialkylammonium.

17. Method as described in claim 1, characterised in that the method is carried out at room temperature.

18. Method as described in claim 1, characterised in that at least in step b) of claim 1 KCl is used as salt.

19. Method as described in claim 1, characterised in that mixtures of different alkali salts and/or alkaline earth salts can also be used in the steps a), c) and d) of claim 1.

20. Method as described in claim 1, characterised in that the nucleic acid mixture is a cleared lysate from prokaryontic cells.

21. Use of the method as described in claim 1, for the purification of plasmid DNA.

22. Use of plasmids obtained by means of a method as described in claim 1 for the preparation of an agent containing plasmid DNA for use in gene therapy or genetic vaccination.
